(19) United States
(12) Patent Application Publication
(10) Pub. No.: US 2009/0082481 A1
(43) Pub. Date: Mar. 26, 2009

(54) Preparation of liquid isocyanurate-modified polymethylene bis(phenylisocyanate) compositions of controlled viscosities

(76) Inventors: James O'Connor, Cheshire, CT (US); Ye Ming, Shanghai (CN)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 11/903,362

(22) Filed: Sep. 21, 2007

(51) Int. Cl. C08G 18/06; C07C 265/12

(52) U.S. Cl. 521/160; 564/248

(57) ABSTRACT

Disclosed is a method for the production of highly stable, liquid isocyanurate-modified PMDI compositions having relatively higher viscosity and a generally comparable functionality, as compared to conventional PMDI. An admixture of the isocyanurate-modified PMDI with conventional PMDI is suitable for use in the manufacture of a variety of polyurethane products, including rigid and flexible foams, coatings, elastomers and sealants. Foams produced using this admixture exhibit properties that are comparable to foams produced from standard polymeric MDI of comparable viscosity that don't contain isocyanurate moieties.

### BACKGROUND OF THE INVENTION

1. Field of the Invention

This invention relates to liquid isocyanurate-modified version of polymeric methylene bis(phenylisocyanate) (PMDI) compositions having viscosities comparable to those of “conventional” PMDI. The preparation methodology used to prepare the isocyanurate-modified PMDI compositions obviates the need for capital equipment expense associated with fractional distillation equipment, thereby providing a capital cost savings as compared to conventional fractional distillation methodology.

This invention also relates to the use of liquid isocyanurate-modified PMDI compositions in making rigid foams that have physical and chemical properties that are comparable to those prepared from “conventional” PMDI.

2. Description of the Material Art

Conventional production processes utilized to produce PMDI typically provide a product mixture containing from about 50% to about 70% of the two ring compounds, with the remainder of the mixture containing 3 or more rings. Although the percentages can vary, and illustrative batch of conventional PMDI product might contain 48% of the two-ring specie, 27% of the three-ring specie, 5% of the four-ring specie, 4% of the five-ring specie, and 16% of higher-ring species, based on the total weight of the PMDI batch. Typically, conventional PMDI product will have a viscosity of 100-300 cps. If the level of two ring isomers is decreased, the viscosity of the mixture increases because the higher-ring components of the mixture have a higher viscosity relative to the two ring compound portion.

Most suppliers of PMDI offer a number of tailored products for the simple reason that different grades of material are utilized in different applications. For example, a product containing essentially all two ring isomers is useful as a starting material in the production of high grade elastomers. For a variety of other applications, including less demanding elastomer applications, as well as rigid and flexible foam applications, product grades containing higher levels of the 3, 4, and 5-ring or higher-ring isomers are advantageously used since these isomers are generally cheaper. Also, the volume demand for products containing the higher-ring isomers is greater in view of the number of end-use applications available.

Heretofore, the products containing a higher level of the 3 ring or greater isomers are generally produced via a fractional distillation process in which the two ring species being MDI is removed leaving a bottom stream with a greater percentage of 3′ ring species and higher. In order for a production plant to operate efficiently, the balance of the pure two ring isocyanates and the higher viscosity, higher functionality isocyanates must be such that enough of each type is produced in an amount sufficient to satisfy the needs of the market. Unfortunately, if the market demands more of the high viscosity, high functionality material, then there has to be a balanced demand for the two ring isocyanate, or otherwise the MDI producer will be left with unwanted isomer product in its stock.

As an alternative to fractional distillation to remove the two-ring isomers from polymeric MDI in order to increase viscosity and functionality, the viscosity of the isocyanate products may be increased by adding non-reactive additives, or by reacting the conventional polymeric MDI products with polyols in order to produce a prepolymer. Both of these approaches have drawbacks. The non-reactive additive does not bond reactively to the final end product, and thus its presence in the end product is detrimental to the strength properties of the product. The prepolymer preparation by reacting with a polyol substantially lowers the isocyanate content of the product which is disadvantageous because the amount of prepolymer needed to react with polyol to produce the finished goods substantially increases.

The prior art discloses that PMDI can be modified using trimerization catalysts in order to produce isocyanurate-modified PMDI. U.S. Pat. Nos. 4,743,627; 4,382,125; and, 5,124,370 disclose the production of a isocyanurate-modified PMDI compositions, and a foam made therefrom. The '627 patent also discloses the addition of the pure two ring specie of MDI to the isocyanurate-modified PMDI compositions to provide a mixture containing at least 60% of the two ring species. The mixture containing such a level of two ring species is said to exhibit reduces color and viscosity, as compared to the isocyanurate-modified PMDI alone.

What is now needed in the marketplace is a process for selectively producing an increased amount of higher viscosity, higher functionality products without incurring a corresponding increase in amount of the pure two-ring MDI specie. The present invention provides an answer to that need.

### SUMMARY OF THE INVENTION

In one aspect, the present invention relates to a method for the production of highly stable, liquid isocyanurate-modified PMDI compositions having relatively higher viscosity and a generally comparable functionality, as compared to conventional PMDI. An admixture of the isocyanurate-modified PMDI with conventional PMDI is suitable for use in the manufacture of a variety of polyurethane products, including rigid and flexible foams, coatings, elastomers and sealants.

In another aspect, the present invention relates to a method for producing liquid isocyanurate-modified PMDI compositions having controlled viscosities from a starting material comprising conventional PMDI.

In yet another aspect, the present invention relates to a composition comprising an admixture of (a) conventional PMDI and (b) isocyanurate-modified PMDI, wherein the weight ratio (a) to (b) is from about 1:2 to about 2:1, and wherein the admixture has a viscosity at 25° C. of from about 400 mPas to about 20,000 mPas; preferably from about 600 mPaS to about 2,500 mPaS; most preferably from about 600 mPaS to about 2,000 mPaS

In yet another aspect, the present invention relates to a composition suitable for use in preparing rigid polyurethane/polyisocyanurate foam, wherein the composition comprises (1) an admixture of (a) conventional PMDI and (b) isocyanurate-modified PMDI, wherein the weight ratio (a) to (b) is from about 1:10 to about 10:1, preferably from about 1:2 to about 10:1, and wherein the admixture has a viscosity at 25° C. of from about 400 mPas to about 20,000 mPas; preferably from about 600 mPaS to about 2,500 mPaS; most preferably from about 600 mPaS to about 2,000 mPaS, (2) a polyol, (3) a blowing agent, (4) a urethane reaction-promoting or isocyanurate reaction-promoting catalyst, (5) a surfactant, and optionally other additives, such as flame retardants. The rigid foams produced using this composition are suitable for a variety of uses, including as thermal insulation material.

The isocyanurate modified PMDI is typically employed in the composition in an amount sufficient to provide an NCO/OH index of from 1 to 4.5.

### BRIEF DESCRIPTION OF THE DRAWINGS

In the accompanying drawings:

FIG. 1 is a graph showing a linear relationship between the % NCO content and the % trimer content;

FIG. 2 is a plot showing the relationship between refractions index and viscosity; and

FIG. 3 is a graph including a set of three curves showing the relationship between viscosity and time for three separate solution employed in the process of the invention.

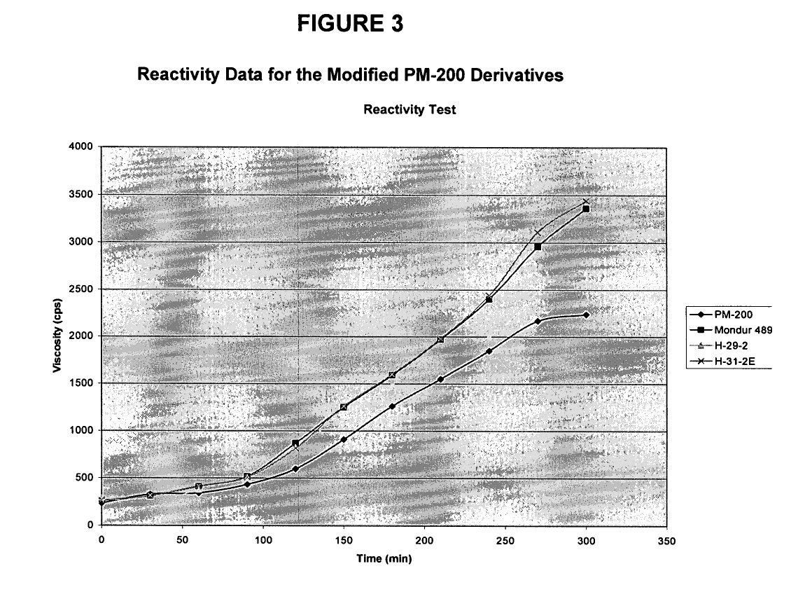

### DETAILED DESCRIPTION OF THE INVENTION

It has now been surprisingly found that an admixture of conventional PMDI and trimerized PMDI provides a liquid product having a controlled viscosity suitable for use in a variety of urethanes applications. The controlled viscosity is comparable to the higher oligomer fraction produced by fractional distillation of conventional PMDI to provide the pure two-ring MDI specie fraction plus the higher oligomer fraction. The admixture of the present invention is produced without using expensive fractional distillation equipment.

The stable isocyanurate-modified PMDI composition of the present invention is prepared by the trimerization in the presence of an effective amount of a trimerization catalyst, of a PMDI to the extent that the conversion to isocyanurate based on the isocyanate content is from about 1 to 50 percent by weight, and the viscosity in mPas at 25° C. is from about 500 to 200,000. After the deactivation of the trimerization catalyst, the isocyanurate-modified polyisocyanate is mixed with the starting material polymeric MDI to achieve viscosities in the range from 400 mPa to 20,000 mPa. The isocyanates of the present invention are useful in the preparation of flexible and rigid foams, comparable to those based on normal polymeric MDI. Although the compositions contain isocyanurate structures, the viscosities are comparable to standard polymeric MDI i.e. from about 300 to 20,000 mPas and the % NOC is essentially the same as the standard polymeric product of the same viscosity. (The higher viscosity products produced by distilling away the two ring isocyanates as well as those produced by the process of this invention, will have a % NCO lower than the lower viscosity products.

That portion of the polyisocyanate which is trimerized is characterized by the presence of the isocyanurate radical in its structure, and in its simplest form may be represented by the formula

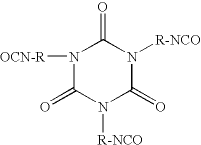

The products of this invention, however, may be complex mixtures in which trimerized and un-trimerized molecules are present and thereby we do not wish to be bound by the structures exemplified above.

The liquid isocyanurate-modified polyisocyanate compositions of the present invention may be prepared by employing well known compounds as trimerization catalysts. Examples of suitable catalysts include (a) organic strong bases, (b) tertiary amine co-catalyst combinations, (c) Friedal Crafts catalysts, (d) basic salts of carboxylic acids, (e) alkali metal oxides, alkali metal alcoholates, alkali metal phenolates, alkali metal hydroxides and alkali metal carbonates, (f) onium compounds from nitrogen, phosphorus, arsenic, antimony, sulfur and selenium, and (g) monosubstituted monocarbamic esters. These include 1,3,5-tris(N,N-dialkylaminoalkyl)-s-hexahydrotriazines; the alkylene oxide and water or carboxylic acid adducts of 1,3,5-tris(N,N-dialkylaminoalkyl)-s-hexahydrotriazines; 2,4,6-tris(dimethylaminomethylphenol); ortho-, para- or a mixture of o- and p-dimethylaminomethylphenol and triethylenediamine or the alkylene oxide and water carboxylic acid adducts thereof, metal carboxylates such as lead octoate, sodium and potassium salts of octano hydroxamic acid, and organic boron containing compounds. Monofunctional alkanols containing from 1 to 24 carbon atoms, epoxides containing 2 to 18 carbon atoms and alkyl carbonates may be used in conjuction with tertiary amines to accelerate the rate of polymerization reaction. The catalysts are present in a catalytically effective amount. Preferably, the concentration of trimerization catalysts that may be employed in the present invention is from about 0.001 part to 20 parts of catalyst per 100 parts of organic polyisocyanate. The temperature ranges which may be employed for the trimerization reaction may be in the range of from about 25° C. to about 230° C., and preferably from about 25° C. to about 120° C.

The preferred trimerization catalyst for this process is: TDH or 1,3,5 tris (N,N-Dimethylaminopropyl)-s hexahydro-5-triazine, (this catalyst is commercially available as Polycat 41 from Air Products).

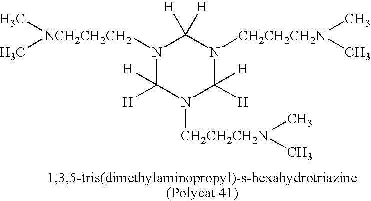

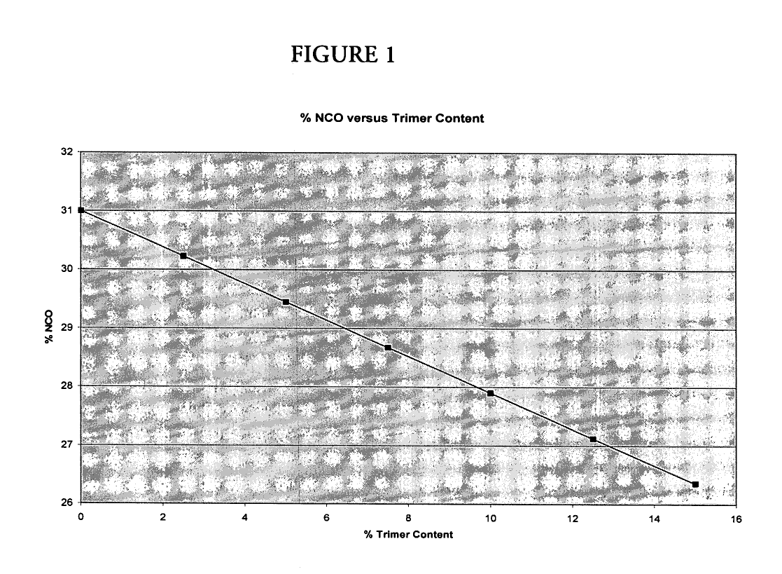

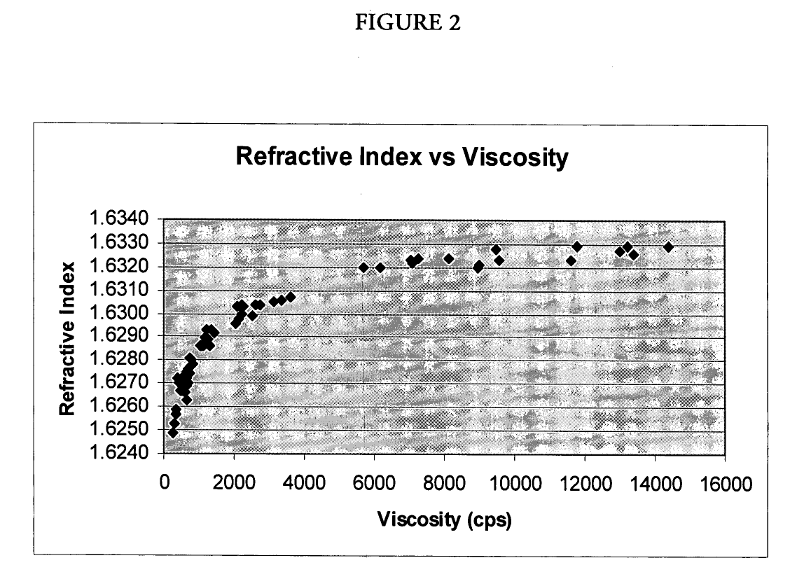

As the trimerization proceeds the % NCO of the isocyanate decreases linearly as shown in FIG. 1 below. A % NCO determination could be used to follow the progress of the reaction. The course of the trimerization reaction can also be followed by the continuous determination of the refractive index. As the trimerization proceeds, the refractive index increases as shown in FIG. 2.

The trimerization catalysts are deactivated after substantially all of the desired polyisocyanate is reacted to form an isocyanurate linkage. The trimerization catalysts are deactivated by the employment of an acid or an acid chloride. The acids may be selected from the group consisting of hydrochloric acid, sulfuric acid, acetic acid, oxalic acid, phosphoric acid, methanesulfonic acid, trifluoromethanesulfonic acid, benzene-, toluene- or xylene sulfonic acids, acid chlorides such as acetyl or benzoyl chloride, and sulfonyl chlorides such as benzene, toluene or xylenesulfonyl chloride, and mixtures thereof may be employed. Another series of deactivators which are alkylating agents such as dimethyl sulfate, o- or p-alkyl toluene sulfonates, and methyl chloride may also be employed. The preferred catalyst deactivators or quenchers are the acid chlorides such as acetyl or benzoyl chloride.

The isocyanurate-modified reaction product has a viscosity of about 2,000 to about 100,000 mPas. After catalyst deactivation, this product is blended with the starting material polymeric MDI such as to achieve a viscosity of the composition at about 400 to 20,000 mPas. The % isocyanate content is comparable to standard polymeric MDI.

Typical trimerization experiments were carried out using the Polycat 41 as the trimer catalyst following the reaction by the change in refractive index. Typically, to achieve a controllable rate and not have excessive catalyst residue to neutralize, the trimerization is carried out at 40-50° C. with about 0.018 g (0.00032 eq-180 ppm) of Polycat 41 per 100 g of polymeric MDI. The Polycat 41 has 6 tertiary amine groups so has an equivalent weight of 57. Initially we quenched or deactivated these reactions by adding benzoyl chloride but we later switched to acetyl chloride. The amount of quench is minimized to keep the hydrolyzable chloride content in the final isocyanate as low as possible, but high enough to insure that the trimerization is completely stopped. Most reactions were terminated with a 5% equivalent excess of acetyl chloride. The equivalent weight of acetyl chloride is 78.5. It is worth noting that this level (1.05 equivalent of quencher per equivalent of catalyst) may be excessive since all polymeric MDI products have a level of background acidity. If too low a level of trimerization catalyst is added initially, the trimerization will either not start or will terminate prematurely. If the trimerization stops, additional trimerization catalyst can be added to restart the reaction. Additionally, when the fresh polymeric MDI is added to the liquid isocyanurate-modified polyisocyanate compositions to achieve the desired lower viscosities, and usually at a weight ratio that is higher than the reaction product that it is added to, the background acidity of this fresh polyisocyanate will also help to neutralize any of the remaining trimer catalyst. This is exemplified in example 8 below.

Another way to minimize the hydrolyzable chloride level is to add epoxy compounds to the polymeric MDI prior to the trimerization. Several patents (U.S. Pat. No. 3,793,362—expired, U.S. Pat. No. 3,925,437—expired and U.S. Pat. No. 5,726,240 claim that by adding the epoxy, the acidity of the MDI is reduced and the reactivity is increased.

It has been found that another advantage of the present invention is that foams with comparable or improved compressive strength properties can be prepared compared to polymeric MDI prepared without the isocyanurate groups present initially. The foams may be prepared as is known in the art by the catalytic reaction of the isocyanurate-modified polyisocyanate with a polyol in the presence of blowing agents, surfactants and the other additives which may be deemed necessary. Noncellular products may also be prepared in the absence of blowing agents as is well known in the art.

The aromatic polyisocyanate (1) isocyanurate modified polymethylene polyphenyl polyisocyanate may be used alone or in combination with other polyisocyanates.

The amount of the isocyanurate modified polymethylene polyphenyl polyisocyanate of component (1) employed in the composition should be sufficient to provide an index of from 1.0 to 4.5. The index is defined as the equivalent ratio of isocyanato groups [NCO groups] to active hydrogen groups in the composition. In addition to component (1), the composition contains (2) a polyol, (3) a blowing agent, (4) a urethane reaction-promoting or isocyanurate reaction-promoting catalyst, (5) a surfactant, and optionally other additives, such as flame retardants.

The polyol (2) is preferably a polyether polyol, a polyester polyol, or mixtures thereof. The polyether polyol is obtained by addition-polymerizing an alkylene oxide (e.g. propylene oxide and/or ethylene oxide) to a reactive starting material, for example, a polyhydric alcohol such as ethylene glycol, propylene glycol, glycerin, trimethylolpropane, pentaerythritol, sorbitol, sucrose and bisphenol A; or an aliphatic amine such as triethanolamine and ethylenediamine, or an aromatic amine such as toluenediamine and methylenedianiline (MDA).

The polyether polyol can be obtained by addition-polymerizing an alkylene oxide to a reactive starting material containing 2-8 reactive hydrogen atoms, preferably 3-8 reactive hydrogen atoms, in the molecule by anionic polymerization in the presence of a catalyst such as alkali hydroxide (e.g. potassium hydroxide and sodium hydroxide) or alkali alcoholate (e.g. potassium methylate and sodium methylate) using a conventionally known method. The polyether polyol can be obtained by adding an alkylene oxide to a reaction starting material due to cationic polymerization in the presence of a catalyst such as Lewis acid (e.g. antimony pentachloride and boron fluoride etherate).

Suitable alkylene oxide includes, for example, tetrahydrofuran, ethylene oxide, 1,3-propylene oxide, 1,2- or 2,3-butylene oxide, 1,2-propylene oxide and styrene oxide. Among them, ethylene oxide and 1,2-propylene oxide are particularly preferred. These alkylene oxides can be used alone or in combination.

The reactive starting material (i.e. initiator) includes, for example, polyhydric alcohols (e.g. ethylene glycol, propylene glycol, glycerin, trimethylolpropane, pentaerythritol, sorbitol, sucrose, and bisphenol A), and mixtures thereof, alkanolamines (e.g. ethanolamine, diethanolamine, N-methyl- and N-ethyl-ethanolamine, N-methyl- and N-ethyl-diethanolamine, triethanolamine), and mixtures thereof. Furthermore, aliphatic amines, aromatic amines, and mixtures thereof, can be used. Examples thereof include ethylenediamine, diethylenetriamine, 1,3-propylenediamine, 1,3- or 1,4-butylenediamine, 1,2-, 1,3-, 1,4-, 1,5- and 1,6-hexamethylenediamine, phenylenediamine, o-toluenediamine, m-toluenediamine, methylenedianiline (MDA), polymethylenedianiline (P-MDA), and mixtures thereof.

As the polyester polyol, there can be used, for example, a polyester polyol such as polyethylene terephthalate, which is prepared from a polycarboxylic acid (e.g. dicarboxylic acid and tricarboxylic acid) and a polyhydric alcohol (e.g. a diol and a triol). Preferred polyester polyols can be produced from a dicarboxylic acid or anhydride having 2 to 12 carbon atoms and a diol having 2 to 12 carbon atoms, preferably 2 to 6 carbon atoms.

The dicarboxylic acid includes, for example, succinic acid, glutaric acid, adipic acid, suberic acid, azelaic acid, sebacic acid, decanedicarboxylic acid, maleic acid, phthalic acid, isophthalic acid and terephthalic acid. In place of the free carboxylic acid, a corresponding carboxylic acid derivative such as dicarboxylic acid monoester or diester with an alcohol having 1 to 4 carbon atoms, or a dicarboxylic anhydride can be used.

As the diol, there can be used, for example, ethylene glycol, diethylene glycol, 1,2- or 1,3-propanediol, dipropylene glycol, 1,4-butanediol, 1,5-pentanediol, 1,6-hexanediol and 1,10-decanediol. As the triol, for example, glycerin and trimethylolpropane can be used. A lactone-based polyester polyol can be also used.

The polyol preferably has a functionality within a range from 2 to 8, and particularly from 2 to 6. Those having a hydroxyl value within a range from 150 to 500 mg KOH/g, preferably from 200 to 500 mg KOH/g, are preferred.

The polyol (2) contains, as a main portion, a polyether polyol or a polyester polyol or a combination of either. The polyol (2) may be composed only of the polyether polyol or polyester polyol, or may be a mixture of the polyether polyol with another polyether polyol and/or a polyester polyol or a polyester polyol with another polyether polyol and/or a polyester polyol.

Any of the blowing agents (3) known in the art for the preparation of rigid polyurethane or urethane-modified polyisocyanurate foams can be used in the process of the present invention. Such blowing agents include water or other carbon dioxide-evolving compounds, or inert low boiling compounds having a boiling point of above −70° C. at atmospheric pressure.

Where water is used as blowing agent, the amount may be selected in known manner to provide foams of the desired density, typical amounts being in the range from 0.05 to 5% by weight based on the total reaction system.

Suitable inert blowing agents include those well known and described in the art, for example, hydrocarbons, dialkyl ethers, alkyl alkanoates, methyl formate, methylal, acetone, aliphatic and cycloaliphatic hydrofluorocarbons, hydrochlorofluorocarbons, chlorofluorocarbons, hydrochlorocarbons and fluorine-containing ethers. Examples of preferred blowing agents include water, isobutane, n-pentane, isopentane, cyclopentane or mixtures thereof; 1,1-dichloro-2-fluoroethane (HCFC 14 lb); 1,1-trifluoro-2-fluoroethane (HFC 134a); chlorodifluoro-methane (HCFC 22); 1,1,1,2,3,3,3-heptafluoropropane (HFC 227ea); 1,1-difluoro-3,3,3-trifluoropropane (HFC 245fa); 1,1,1,3,3-pentafluorobutane (HFC 365mfc.); 1,1,1,3,3-pentafluoropropane (HCFC 245fa), and combinations thereof. Particular mention may be made of blowing agent mixtures as described in PCT Patent Publication No. 96/12758, incorporated herein by reference, for manufacturing low density, dimensionally stable rigid foam. These blowing agent mixtures generally comprise at least 3 and preferably at least 4 components of which preferably at least one is a (cyclo)alkane (preferably of 5 or 6 carbon atoms) and/or acetone.

The blowing agents are employed in an amount sufficient to give the resultant foam the desired bulk density which is generally in the range 15 to 70 kg/m.sup.3, preferably 20 to 50 kg/m.sup.3, most preferably 25 to 40 kg/m.sup.3. Typical amounts of blowing agents are in the range 2 to 25% by weight based on the total reaction system.

When a blowing agent has a boiling point at or below ambient it is maintained under pressure until mixed with the other components. Alternatively, it can be maintained at subambient temperatures until mixed with the other components.

The catalysts (3) which are customary in polyurethane and polyisocyanurate chemistry can be used in the method according to the invention. Examples of catalysts of this type include: triethylenediamine, N,N-dimethylcyclohexylamine, tetramethylenediamine, 1-methyl4-dimethyl-aminoethylpiperazine, triethylamine, tributylamine, dimethylbenzylamine, N,N′,N″-tris-(dimethylaminopropyl)-hexahydrotriazine, dimethylamino-propylformamide, N,N,N′,N′-tetramethylethylenediamine, N,N,N′,N′-tetramethylbutanediamine, tetramethylhexanediamine, pentamethyldiethylenetriamine, tetramethyldiaminoethyl ether, dimethylpiperazine, 1,2-dimethylimidazole, 1-aza-bicyclo-(3,3,0)-octane, bis-(dimethylaminopropyl)-urea, N-methylmorpholine, N-ethylmorpholine, N-cyclohexylmorpholine, 2,3-dimethyl-3,4,5,6,-tetrahydropyrimidine, triethanolamine, diethanolamine, triisopropanolamine, N-methyldiethanolamine, N-ethyldiethanolamine, dimethylethanolamine, tin(II) acetate, tin(II) octoate, tin(II) ethylhexoate, tin(II) laurate, dibutyltin diacetate, dibutyltin dilaurate, dibutyltin maleate, dioctyltin diacetate, tris-(N,N-dimethyl-aminopropyl)-s-hexahydrotriazine, tetramethylammnonium hydroxide, sodium acetate, potassium acetate, sodium octoate, potassium octoate, sodium hydroxide, or mixtures of these or similar catalysts.

At least one surfactant (4) is also employed. Nonionic surfactants are preferred. Nonionic surface active agents prepared by the sequential addition of propylene oxide and then ethylene oxide to propylene glycol in the solid or liquid organo silicones have been found particularly desirable. Other surfactants which are usable, although not preferred, include polyethylene glycol ethers of long chain alcohols, tertiary amine or alkanol amine salts of long chain alkyl acid sulfate esters, alkyl sulfonic esters, and alkyl aryl sulfonic acids.

At least one flame retardant is optionally employed. *Examples of suitable flameproofing agents are tricresyl phosphate, tris(2-chloroethyl) phosphate, tris(2-chloropropyl) phosphate, and tris(2,3dibromopropyl) phosphate. A suitable flame retardant in compositions of the present invention comprises FYROL.RTM. PCF, which is a tris(chloro propyl)phosphate commercially available from Albright & Wilson.

As an alternative or addition to the above-mentioned halogen-substituted phosphates, it is also possible to use inorganic or organic flameproofing agents, such as red phosphorus, aluminum oxide hydrate, antimony trioxide, arsenic oxide, ammonium polyphosphate (Exolit.RTM.) and calcium sulfate, expandable graphite or cyanuric acid derivatives, e.g., melamine, or mixtures of two or more flameproofing agents, e.g., ammonium polyphosphates and melamine, and, if desired, corn starch, or ammonium polyphosphate, melamine, and expandable graphite andlor, if desired, aromatic polyesters, in order to flameproof the polyisocyanate polyaddition products. In general, from 2 to 50 parts by weight, preferably from 5 to 25 parts by weight, of said flameproofing agents may be used per 100 parts by weight of the polyol component of the composition.

Other optional may also suitably be employed. These include one or more of the following: foam stabilisers, cell regulators, reaction inhibitors, dyes, fillers, fungistatically and/or bacteriostatically active substances. Details relating to the manner of use and mode of action of these additives are described in Kunststoff-Handbuch, volume VII, edited by Vieweg and Hochtlen, Carl Hanser Verlag, Munich 1966, for example on pages 121 to 205, and 2nd edition 1983, edited by G. Oertel (Carl Hanser Verlag, Munich), incorporated herein by reference in its entirety.

The following examples are offered to illustrate various aspects of the invention. Those skilled in the art understand that there are many possible modifications and the examples are not to be construed as limiting the scope and spirit of the invention.

### EXAMPLES 1-5

General Procedure—Into a 3 neck flask equipped with a mechanical stirrer, a thermometer, a gas inlet tube was added the polymeric MDI—PM-200 (Yantai Wanawha Polyurethanes Inc.). The contents were heated either to 40 or 50° C. and then the catalyst Polycat 41 catalyst was added. The progress of the reaction was monitored by following the change in refractive index (RI) with time. The reaction was terminated with acetyl chloride and the mixture was stirred for another 60 minutes at 40 or 50° C. Next the product was removed, divided into either two or four equal portions and mixed with different levels of starting material PM-200 with one remaining undiluted isocyanarate modified PM-200 to follow the stability of it as a function of time. The results of these reactions are shown in Table 1. Note that the measurements for viscosity and refractive index are for the most part essentially constant as a function of time. There are some variations in the measured values and these can be explained by looking at the temperature when the measurement was made. The measurements were made at ambient conditions and we typically see temperature fluctuations of ±2° C. The viscosity is higher when temperatures are lower and the refractive index will also decrease by approximately 0.0001 units for every 1° C. drop in temperature. One other effect was observed when these measurements were taken and that is, some of the samples built a surface skin over the period of time when these measurements were made and this is probably due to a surface reaction of the isocyanaruate modified polymeric MDI with ambient moisture. This effect would also cause some slight variation in the measured readings. The products produced by this process are stable especially with the level of quencher used for these runs.

Note also the % NCO. The starting material has a free NCO of 30.66 or 30.98%. Depending on the degree of trimerization, the % NCO drops (see G-93-1, G-95-1, H-25-1 and H-27-1), but when the starting material polymeric is added back to the isocyanurate modified polymeric MDI to achieve a viscosity of ˜600 cps, the % NCO is >30.1 a value similar to the Mondur 483 (30.5).

TABLE 1
Trimerization Results Examples 1-5

| | PM | | | | | | | | | | | |
|---|---|---|---|---|---|---|---|---|---|---|---|---|
| | 200 (g) | | | Reaction | acetyl | Trimer/ | | | | | | |
| | lot (1) | catalyst | | Time | chloride | PM-200 | Measurement | Refractive | | viscosity | | |
| Run # | lot (2) | (g) | T° C. | (min) | (g) | ratio | time (day) | Index | temp⁽° C.) | (cps) | temp⁽° C.) | % NCO |
| PM 200 | | | | | | 0/100 | 1 | 1.6253 | 22.5 | 292 | 23 | 30.66 |
| lot 1 | | | | | | | | | | | | |
| PM 200 | | | | | | 0/100 | Initial | 1.6250 | 22.2 | 276 | 22 | 30.98 |
| lot 2 | | | | | | | | | | | | |
| G-89-1 | 700 (1) | 0.1232 | 40 | 105 | 0.1848 | 100/0 | Initial | 1.6320 | 22.5 | | | |
| | | | | | | | 1 | 1.6320 | 22.5 | | | |
| | | | | | | | 7 | 1.6320 | 22.2 | 6180 | 23 | |
| G-89-2 | | | | | | 75/25 | Initial | 1.6303 | 22.6 | | | |
| | | | | | | | 1 | 1.6304 | 21.8 | 2760 | 23 | |
| | | | | | | | 7 | 1.6304 | 21.8 | 2620 | 23 | |
| G-89-3 | | | | | | 50/50 | Initial | 1.6284 | 22.8 | | | |
| | | | | | | | 1 | 1.6286 | 22.2 | 1120 | 23 | |
| | | | | | | | 7 | 1.6286 | 21.7 | 1080 | 23 | |
| G-89-4 | | | | | | 25/75 | Initial | 1.6264 | 23.0 | | | 30.14 |
| | | | | | | | 7 | 1.6272 | 21.7 | 590 | 23 | |
| G-93-1 | 700 (2) | 0.1266 | 50 | 130 | 0.1783 | 100/0 | Initial | 1.6322 | 24.0 | | | 27.92 |
| | | | | | | | 1 | 1.6323 | 23.0 | 11,600 | 22 | |
| | | | | | | | 28 | 1.6329 | 21.8 | 11,800 | 23 | |
| G-93-2 | | | | | | 75/25 | Initial | 1.6298 | 24.2 | | | |
| | | | | | | | 1 | 1.6306 | 22.7 | 3360 | 22 | |
| G-93-3 | | | | | | 50/50 | Initial | 1.6275 | 24.6 | | | 29.51 |
| | | | | | | | 1 | 1.6286 | 22.7 | 1300 | 22 | |
| G-93-4 | | | | | | 25/75 | Initial | 1.6256 | 24.8 | | | |
| | | | | | | | 1 | 1.6267 | 22.7 | 528 | 22 | |
| G-95-1 | 1100 (2) | 0.2005 | 50 | 120 | 0.2868 | 100/0 | Initial | 1.6324 | 23.3 | | | 27.96 |
| | | | | | | | 1 | 1.6327 | 22.5 | 13,000 | 22 | |
| | | | | | | | 27 | 1.6329 | 22.0 | 13,200 | 23 | |
| G-95-2 | | | | | | 64/36 | Initial | 1.6293 | 23.1 | | | 29.28 |
| | | | | | | | 1 | 1.6299 | 22.5 | 2540 | 22 | |
| | | | | | | | 2 | 1.6300 | 21.9 | 2200 | 21.6 | |
| H-25-1 | 1200 (2) | 0.2134 | 40 | 165 | 0.29 | 100/0 | Initial | 1.6321 | 24.7 | | | 28.34 |
| | | | | | | | 1 | 1.6321 | 23.1 | 9020 | 22 | |
| | | | | | | | 28 | 1.6324 | 22.3 | 8150 | 23 | |
| H-25-4 | | | | | | 32.5/67.5 | Initial | 1.6263 | 24.7 | 651 | 24 | 30.12 |
| | | | | | | | 1 | 1.6270 | 22.6 | 708 | 22 | |
| | | | | | | | 28 | 1.6274 | 22.2 | 735 | 23 | |
| H-27-1** | 1000 (2) | 0.1778 | 40 | 150 | 0.2599 | 100/0 | Initial | 1.6321 | 23.6 | | | 27.84 |
| | | | | | | | 1 | 1.6326 | 22.2 | 13,400 | 23 | |
| | | | | | | | 27 | 1.6329 | 22.3 | 14,400 | 23 | |
| H-27-2 | | | | | | 27.8/72.2 | Initial | 1.6266 | 23.3 | 618 | 23 | 30.14 |
| | | | | | | | 1 | 1.6269 | 22.2 | 638 | 23 | |
| | | | | | | | 27 | 1.6272 | 22.3 | 671 | 23 | |

**1.2996 g 3,4-epoxycyclohexyl methyl-3,4-epoxycyclohexane carboxylate added to the PM 200

### EXAMPLE 6

H-29-2—Into a 3 liter 3 neck flask equipped with a mechanical stirrer, a thermometer, a gas inlet tube was added 1200 g of PM-200. The contents were heated to 50° C. and then 0.220 g of Polycat 41 (183 ppm) catalyst was added. The progress of the reaction was monitored by following the change in refractive index (RI) with time.

| Time | RI |
|---|---|
| 9:55 | |
| 10:15 | 1.6269/22.1° C. |
| 10:35 | 1.6293/22.6° C. |
| 10:45 | 1.6303/22.9° C. |
| 10:55 | 1.6308/23.1° C. |
| 11:05 | 1.6311/23.2° C. |
| 11:15 | 1.6316/23.5° C. |
| 11:25 | 1.6324/23.6° C. |

The reaction was quenched with 0.320 g (267 ppm) of acetyl chloride and the mixture was stirred for another 60 minutes at 50° C. RI=1.6322/24° C.). Next 605 g product was removed and mixed with 1254 g of PM-200 starting material (H-29-2). The remaining undiluted trimerized PM-200 (H-29-1) was left to follow the stability as a function of time.

The viscosity and refractive index of the diluted and undiluted samples were determined as a function of time.

| Time | | Sample | RI | viscosity (cps) |
|---|---|---|---|---|
| 0 | | 29-2 | 1.6270/24.0° C. | — |
| 1 | day | 29-2 | 1.6271/23.6° C. | 785/23° C. |
| 5 | day | 29-2 | 1.6276/21.3° C. | 721/24° C. |
| 13 | day | 29-2 | | 748/23° C. |
| 20 | day | 29-2 | | 762/23° C. |
| 1 | day | 29-1 | 1.6326/23.7° C. | 12800/23° C. |
| 2 | day | 29-1 | 1.6329/23.6° C. | 13300/23° C. |
| 5 | day | 29-1 | 1.6333/21.5° C. | 4000/24° C. |
| 13 | day | 29-1 | | 12900/23° C. |

The results indicate that both the undiluted and diluted samples are stable.

### EXAMPLE 7

H-31-2E—Into a 3 liter 3 neck flask equipped with a mechanical stirrer, a thermometer, a gas inlet tube was added 1200 g of PM-200 and 1.56 g of 3,4-epoxycyclohexylmethyl 3,4-epoxycyclohexane carboxylate. The contents were heated to 65° C. and held there for 1 hour. Next the mixture was cooled to 50° C. and 0.219 g of Polycat 41 (182 ppm) catalyst was added. The progress of the reaction was monitored by following the change in refractive index (RI) with time.

| Time | RI |
|---|---|
| 10:47 | — |
| 11:23 | 1.6278/24.4° C. |
| 11:47 | 1.6301/24.4° C. |
| 12:07 | 1.6315/24.4° C. |
| 12:23 | 1.6321/24.4° C. |
| 11:15 | 1.6316/23.5° C. |
| 11:28 | — |

The reaction was quenched with 0.316 g (265 ppm) of acetyl chloride and the mixture was stirred for another 20 minutes while cooling to 45° C. Next 600 g product was removed and mixed with 1474 g of PM-200 starting material (H-31-2E). The remaining undiluted trimerized PM-200 (H-31-1E) was left to follow the stability as a function of time.

The viscosity and refractive index of the diluted and undiluted were determined as a function of time.

| Time | | Sample | RI | viscosity (cps) |
|---|---|---|---|---|
| 0 | | 31-2E | 1.6264/24.5° C. | — |
| 1 | day | 31-2E | 1.6272/22.5° C. | 681/23° C. |
| 4 | day | 31-2E | 1.6276/21.4° C. | 725/24° C. |
| 12 | day | 31-2E | | 721/23° C. |
| 19 | day | 31-2E | | 728/23° C. |
| 1 | day | 31-1E | 1.6329/23.5° C. | 19200/23° C. |
| 4 | day | 31-1E | 1.6335/21.6° C. | 16900/24° C. |
| 12 | day | 31-1E | | 16300/23° C. |

The results indicate that both the undiluted and diluted samples are stable.

The reactivity of the two isocyanaruate modified PM-200 isocyanates(H-29-2 and H-31-2E) was compared in a reactivity test to the starting material PM-200 and Mondur 489. This test was performed by mixing an equivalent amount of each isocyanate with a 1000 molecular weight ethylene oxide capped prolypropylene glycol polyol (Poly G 55-112). The build in viscosity was measured as a function of time. One would expect that the higher functionality isocyanates would build viscosity at a higher rate than those with lower functionality. High acidity or hydrolyzable chloride content may mitigate this effect. The results are shown in FIG. 1.

FIG. 1—Reactivity Data for the Modified PM-200 Derivatives

From the plot one can see that the two isocyanurate modified PM-200 derivatives and the high functionality Mondur control as expected all build viscosity at a higher rate than the PM-200. The epoxy modified derivative (H-31-2E) almost overlays the Mondur control in reactivity. The H-29-2 is more reactive than the PM-200 starting material but slightly lower than the control and epoxy modified isocyanate. This could be related to the higher hydrolyzable chloride content due to the quench.

### EXAMPLE 8

H-37-2—The H-37-2 sample was produced by a different process. We started the run with about ½ the trimer catalyst used for earlier runs and a reaction temperature of 60° C. The reaction was sluggish and more catalyst added and a third increment was added later (total catalyst added=188.5 ppm). This is the catalyst level that we ordinarily use for the trimerization but when added incrementally in this manner, the residual acidity and hydrolyzable chlorides tend to deactivate the catalyst. This trimerization stopped without quenching at a refractive index of 1.6305 rather than the desired 1.6325. It did however reach a refractive index=1.6332 after standing at room temperature for two days. (a threshold catalyst concentration is required in trimerizations because the acidity/hydrolyzable chlorides of the starting isocyanate neutralizes some catalyst).

Into a 3 liter 3 neck flask equipped with a mechanical stirrer, a thermometer, a gas inlet tube was added 700 g of PM-200. The contents were heated to 60° C. and then 0.108 g of Polycat 41 (154 ppm) catalyst was added. The progress of the reaction was monitored by following the change in refractive index (RI) with time. Because the reaction was sluggish, two other increments of catalyst were added (see below).

| Time | RI |
|---|---|
| 10:32 | 154 ppm catalyst added |
| 11:06 | 1.6271/23.2° C. |
| 11:33 | 1.6278/23.9° C. |
| 12:02 | 1.6282/24.3° C. |
| 1:30 | 1.6287/24.4° C. (add 0.0142 g cat-174.5 ppm total) |
| 2:00 | 1.6293/24.5° C. |
| 2:30 | 1.6297/24.5° C. |
| 3:00 | 1.6300/24.4° C. |
| 3:30 | 1.6300/25.1° C. (add 0.0098 g cat-188.5 ppm total) |
| 4:00 | 1.6305/25.4° C. |
| 4:30 | 1.6305/25.5° C. |

Since the reaction stopped on its own, no quench was added. Next 100 g of product was diluted with 250 g of PM-200 (H-37-2) The remaining undiluted trimerized PM-200 (H-37-1) was left to follow the stability as a function of time.

The viscosity and refractive index of the diluted and undiluted were determined as a function of time.

| Time | | Sample | RI | viscosity (cps) |
|---|---|---|---|---|
| 2 | day | 37-2 | 1.6268/23.7° C. | 745/22.5° C. |
| 16 | day | 37-2 | 1.6273/23.3° C. | 730/21.6° C. |
| 0 | | 37-2 | 1.6305/25.5° C. | — |
| 2 | day | 37-1 | 1.6332/22.8° C. | 12300/22.5° C. |

Sample 37-2 is stable after 16 days even with no quench added. We attribute this to the fact that most if not all the catalyst was quenched during the prolonged reaction with residual acidity and hydrolyzable chlorides in the PM-200. In addition to this, when the high viscosity reaction product is diluted down to the 700 cps viscosity, additional acidity/hydrolyzable hydrolyzable chloride in the virgin polymeric isocyanate, further quenches or neutralizes any remaining catalyst in the product.

We took the unquenched trimer and diluted it down to ˜700 cps.

In order to assess how significant a role the excess hydrolyzable chloride content has on foam properties, in addition to the three isocyanurate modified polymeric MDI isocyanates described in examples 6-8, 185 ppm acetyl chloride was added to the starting material PM-200 and this was our 4^{th} isocyanate (PM-200 Ac) to evaluate in foaming studies.

We made up a series of foams at 3 indicies (2.5, 2.0 and 1.5) using the PM-200 and Mondur 489 as controls along with the four different isocyanates described above. One other control was added in which with 185 ppm of acetyl chloride was added to virgin PM-200. The actual formulations tested are shown in Tables 3, 4 and 5; Table 3-1.5 index, Table 4-2.0 index and Table 6-2.5 index.

TABLE 3
1.5 Index Formulations for Compressive Strength Testing

| | 51-2 | 51-3 | 51-6 | 51-5 | 53-1 | 52-1 |
|---|---|---|---|---|---|---|
| Terol 925 | 100 | 100 | 100 | 100 | 100 | 100 |
| Polycat 46 | 2.0 | 2.0 | 2.0 | 2.0 | 2.0 | 2.0 |
| Curithane 52 | 2.0 | 2.0 | 2.0 | 2.0 | 2.0 | 2.0 |
| DC-193 | 1.0 | 1.0 | 1.0 | 1.0 | 1.0 | 1.0 |
| Water | 1.50 | 1.50 | 1.50 | 1.50 | 1.50 | 1.50 |
| Enovate | 23 | 26 | 24 | 25 | 23 | 25 |
| 3000 | | | | | | |
| PM 200 | 140.3 | | | | | |
| H-29-2 | | 145.9 | | | | |
| H-31-2E | | | 145.9 | | | |
| Mondur 489 | | | | 143.8 | | |
| PM 200 Ac | | | | | 140.3 | |
| H-37-2 | | | | | | 145.9 |

TABLE 4
2.0 Index Formulations for Compressive Strength Testing

| | 49-3 | 49-2 | 51-1 | 49-4 | 53-2 | 52-4 |
|---|---|---|---|---|---|---|
| Terol 925 | 100 | 100 | 100 | 100 | 100 | 100 |
| Polycat 46 | 2.0 | 2.0 | 2.0 | 2.0 | 2.0 | 2.0 |
| Curithane 52 | 2.0 | 2.0 | 2.0 | 2.0 | 2.0 | 2.0 |
| DC-193 | 1.0 | 1.0 | 1.0 | 1.0 | 1.0 | 1.0 |
| Water | 1.50 | 1.50 | 1.50 | 1.50 | 1.50 | 1.50 |
| Enovate | 28 | 33 | 31 | 32 | 28 | 31 |
| 3000 | | | | | | |
| PM 200 | 187.0 | | | | | |
| H-29-2 | | 194.5 | | | | |
| H-31-2E | | | 194.5 | | | |
| Mondur 489 | | | | 191.7 | | |
| PM 200 Ac | | | | | 187.0 | |
| H-37-2 | | | | | | 194.5 |

TABLE 5
2.5 Index Formulations for Compressive Strength Testing

| | 45-3 | 48-2 | 48-3 | 48-5 | 53-3 | 52-3 |
|---|---|---|---|---|---|---|
| Terol 925 | 100 | 100 | 100 | 100 | 100 | 100 |
| Polycat 46 | 2.0 | 2.0 | 2.0 | 2.0 | 2.0 | 2.0 |
| Curithane 52 | 2.0 | 2.0 | 2.0 | 2.0 | 2.0 | 2.0 |
| DC-193 | 1.0 | 1.0 | 1.0 | 1.0 | 1.0 | 1.0 |
| Water | 1.50 | 1.50 | 1.50 | 1.50 | 1.50 | 1.50 |
| Enovate 3000 | 30 | 34 | 34 | 36 | 33.0 | 34 |
| PM 200 | 223.8 | | | | | |
| H-29-2 | | 243.1 | | | | |
| H-31-2E | | | 243.1 | | | |
| Mondur 489 | | | | 239.6 | | |
| PM 200 Ac | | | | | 223.8 | |
| H-37-2 | | | | | | 243.1 |

The samples were tested and the results are shown in Tables 6. This table is included at the end of the report. The foam samples were produced in 32 oz cups (˜1 liter) and the blowing agent adjusted to achieve a 2 lb/ft³ (32 kg/m³) density. For most foams the 2.0±0.1 lb/ft³ densities were achieved, but there are some that are above and below this range so consideration should be given to the density variations when examining the results. Each density and compressive strength value is the average value from 3-5 separate samples. For the most part, there was good agreement among the different samples of each specific foam but occasionally the value obtained for one in the group deviated fairly significantly from the others but these values were still averaged in with the other results.

Essentially all the foams in Table 5 at all three indicies are anisotropic, that is, the cells are elongated in the direction of rise leading to higher compressive strengths in the direction parallel to rise than in the direction perpendicular to rise. Looking first at the 2.5 index foams, the highest compressive strength values were obtained for the two isocyanurate modified polymeric MDI and control pMDI foams. The Mondur 489 foam was slightly inferior and the epoxy modified Trimer foam exhibited the worst compressive properties. At 2.0 index, the Mondur and the acid chloride modified foams had superior properties while all the other foams had similar performance. At 1.5 index, the highest compressive strength values were obtained for the quenched and unquenched isocyanurate modified polymeric MDI derived foams.

TABLE 6
Compressive Strength Properties of Trimer Modified PM-200

| | | | Compressive | | | | | Compressive | | | | | Compressive | |
|---|---|---|---|---|---|---|---|---|---|---|---|---|---|---|
| | Density | | Strength | | | Density | | Strength | | | Density | | Strength | |
| | | | | | | | kg/ | | | | | kg/ | | |
| 2.5 Index | lb/ft [symbol] | kg/m [symbol] | lb/ln [symbol] | kPa | 2.0 Index | lb/ft [symbol] | m [symbol] | lb/ln [symbol] | kPa | 1.5 Index | lb/ft [symbol] | m [symbol] | lb/ln [symbol] | kPa |
| 47-3 | | | | | 49-3 | | | | | 51-2 (M-200) | | | | |
| (M-200) | | | | | (M-200) | | | | | | | | | |
| parallel \|\| | 2.14 | 34.3 | 35.2 | 242.5 | parallel \|\| | 1.95 | 31.2 | 21.0 | 145.2 | parallel \|\| | 1.8 | 28.8 | 14.9 | 102 |
| perpen- | 1.90 | 30.4 | 22.7 | 156.9 | perpen- | 2.02 | 32.4 | 14.3 | 98.7 | perpendicular⊥ | 1.82 | 29.2 | 11.1 | 76. [symbol] |
| dicular⊥ | | | | | dicular⊥ | | | | | | | | | |
| 48-2 | | | | | 49-2 | | | | | 51-3 (H-29-2) | | | | |
| (H-29-2) | | | | | (H-29-2) | | | | | | | | | |
| parallel \|\| | 2.1 | 33.6 | 35.4 | 244.4 | parallel \|\| | 1.96 | 31.4 | 18.1 | 124.6 | parallel \|\| | 2.03 | 32.5 | 34.4 | 236 |
| perpen- | 2.01 | 32.1 | 18.4 | 127.2 | perpen- | 1.97 | 31.5 | 17.2 | 119.0 | perpendicular⊥ | 1.83 | 29.3 | 16.6 | 114 |
| dicular⊥ | | | | | dicular⊥ | | | | | | | | | |
| 48-3 | | | | | 51-1 | | | | | 51-6 (H-31-2E) | | | | |
| (H-31-2E) | | | | | (H-31-2E) | | | | | | | | | |
| parallel \|\| | 2.14 | 34.3 | 15.4 | 106.1 | parallel \|\| | 2.09 | 33.4 | 20.9 | 143.8 | parallel \|\| | 1.98 | 31.7 | 30.4 | 209 |
| perpen- | 2.14 | 34.3 | 45.4 | 106.1 | perpen- | 2.05 | 32.9 | 15.2 | 105.1 | perpendicular⊥ | 2.03 | 32.5 | 13.6 | 93. [symbol] |
| dicular⊥ | | | | | dicular⊥ | | | | | | | | | |
| [symbol] 8-5 | | | | | 49-4 | | | | | 51-5 (Mondur 489) | | | | |
| (Mondur | | | | | (Mondur 489) | | | | | | | | | |
| 489) | | | | | | | | | | | | | | |
| parallel \|\| | 2.14 | 34.3 | 31.1 | 214.8 | parallel \|\| | 1.92 | 30.8 | 24.8 | 171.1 | parallel \|\| | 1.88 | 30.1 | 18.3 | 126 |
| perpen- | 2.16 | 34.7 | 24.3 | 167.3 | perpen- | 1.99 | 31.9 | 18.4 | 126.7 | perpendicular⊥ | 1.88 | 30.1 | 13.6 | 93. [symbol] |
| dicular⊥ | | | | | dicular⊥ | | | | | | | | | |
| 52-3 | | | | | 52-4 | | | | | 52-1 (H-37-2) | | | | |
| (H-37-2) | | | | | (H37-2) | | | | | | | | | |
| parallel \|\| | 2.02 | 32.4 | 34.3 | 236.4 | parallel \|\| | 1.69 | 27.0 | 20.7 | 142.6 | parallel \|\| | 1.98 | 31.7 | 31.9 | 220 |
| perpen- | 1.97 | 31.5 | 27.5 | 189.7 | perpen- | 1.65 | 26.4 | 17.3 | 119.3 | perpendicular⊥ | 1.84 | 29.4 | 15.4 | 106 |
| dicular⊥ | | | | | dicular⊥ | | | | | | | | | |
| 53-3 | | | | | 53-2 | | | | | 53-1 (PM-200 Ac) | | | | |
| (PM-200 | | | | | (PM-200 Ac) | | | | | | | | | |
| Ac) | | | | | | | | | | | | | | |
| parallel \|\| | 1.9 | 30.4 | 29.5 | 203.4 | parallel \|\| | 1.82 | 29.1 | 25.0 | 172.3 | parallel \|\| | 1.78 | 28.6 | 25.9 | 178 |
| perpen- | 1.83 | 29.2 | 21.9 | 150.9 | perpen- | 1.77 | 28.4 | 15.3 | 105.4 | perpendicular⊥ | 1.62 | 25.9 | 14.2 | 97. [symbol] |
| dicular⊥ | | | | | dicular⊥ | | | | | | | | | |

[symbol]

indicates data missing or illegible when filed

What is claimed:

1. A method for producing a liquid, isocyanurate-modified PMDI having controlled viscosity comprising the steps of:
(a) trimerizing conventional PMDI in the presence of a catalytically effective amount of a trimerization catalyst to produce isocyanurate-containing PMDI having a viscosity at 25° C. of from about 2,000 mPas to ABOUT 200,000 mPas;
(b) deactivating the trimerization catalyst to provide a mixture containing isocyanurate-modified PMDI and deactivated trimerization catalyst; and,
(c) admixing the mixture from step (b) with an amount of conventional PMDI sufficient to provide an admixture having a viscosity at 25° C. of from about 400 mPas to about 20,000 mPas, and a free NCO content comparable to that of conventional PMDI.

2. The method of claim 1 wherein the product of step (a) has a viscosity at 25° C. of from about 2,000 to 50,000 mPas.

3. The method of claim 1 wherein the isocyanurate-containing PMDI of step (a) has a viscosity at 25° C. of from about 5,000 to 20,000 mPas.

4. The method of claim 1 wherein the admixture of step (c) has a viscosity at 25° C. of from 600 to 2,500 mPas.

5. The method of claim 1 wherein the admixture of step (c) has a viscosity at 25° C. of from 600 to 2,000 mPas.

6. The admixture produced by the method of claim 1.

7. A composition comprising an admixture of (a) conventional PMDI and (b) isocyanurate-modified PMDI, wherein the weight ratio (a) to (b) is from about 1:10 to about 10:1, and wherein the admixture has a viscosity at 25° C. of from about 400 mPas to about 20,000 mPas.

8. The composition of claim 7 wherein the admixture has a viscosity at 25° C. of from about 600 mPaS to about 2,500 mPaS.

9. The composition of claim 7 wherein the admixture has a viscosity at 25° C. of from about 600 mPaS to about 2,000 mPaS.

10. A composition suitable for use in preparing rigid polyurethane/polyisocyanurate foam, wherein the composition comprises (1) an admixture of (a) conventional PMDI and (b) isocyanurate-modified PMDI, wherein the weight ratio (a) to (b) is from about 1:10 to about 10:1, and wherein the admixture has a viscosity at 25° C. of from about 400 mPas to about 20,000 mPas;, (2) a polyol (3) a blowing agent, (4) a urethane reaction-promoting catalyst, (5) a surfactant, and optionally (6) a flame retardant.

11. The composition of claim 10 wherein component (1) has an NCO index within the range of from 1 to 4.5.

12. A method of preparing a rigid polyurethane/polyisocyanurate foam comprising reacting in a reaction vessel the composition of claim 10.

13. A rigid foam prepared by the method of claim 12 being a thermally insulating foam.

14. The composition of claim 10 wherein the admixture of component (1) has a viscosity of from about 600 mPaS to about 2,500 mPaS.

15. The composition of claim 10 wherein the admixture of component (1) has a viscosity of from about 600 mPaS to about 2,000 mPaS.
